(19) United States
(12) Patent Application Publication
(10) Pub. No.: US 2005/0090840 A1
(43) Pub. Date: Apr. 28, 2005

(54) Tissue repair

(75) Inventors: Peter Gerbino, Wellesley, MA (US); Paul Kendrick, South River, NJ (US); William W. Smith, Rye, NH (US); David G. Norman, Worcester, MA (US); Gregory R. Dion, Tucson, AZ (US); Manuel D. Goulart JR., Gloucester, MA (US)

Correspondence Address: FISH & RICHARDSON PC, 225 FRANKLIN ST, BOSTON, MA, 02110, US

(73) Assignees: The Children's Medical Center Corporation, Boston, MA (US); Northeastern University, Boston, MA (US)

(21) Appl. No.: 10/861,250

(22) Filed: Jun. 3, 2004

Related U.S. Application Data
(63) Continuation of application No. PCT/US02/38536, filed on Dec. 3, 2002.
(60) Provisional application No. 60/380,724, filed on May 15, 2002.
(60) Provisional application No. 60/336,802, filed on Dec. 4, 2001.

(51) Int. Cl. A61B 17/10; A61B 17/04; A61B 17/12

(52) U.S. Cl. 606/148; 606/144

(57) ABSTRACT

The proposed meniscus suturing machine provides quick, strong, reliable sutures to torn medial menisci. An electronically controlled pneumatic system powers the suturing process. The meniscus suturing machine utilizes the chain stitch, a common sewing stitch, and applies it to the repair of torn menisci using only a single thread. The designed device is simple to use, with an on/off switch and fire button. While the device is simple, it has internal fault protection devices such as an automatic emergency shutoff and suture recovery loop. The device also comes apart easily, with a disposable needle-thread system. An autoclave sterilizes the remaining portion of the device. The initial application of the design is to suture medial menisci, but modifications will allow the suturing device to accommodate other areas too, such as the lateral meniscus. The benefits of the suturing device include a reduction in operating time and complexity of meniscus repairs.

This application claims the benefit of priority from provisional U.S. patent applications Ser. Nos. 60/336,802, filed Dec. 4, 2001, and 60/380,724, filed May 15, 2002, both of which are incorporated here in their entireties.

This description relates to tissue repair.

Stitching of body tissue is a step in many medical procedures.

Stitching (also called suturing) is used, for example, for meniscal repair in the knee and is also used in other types of knee and shoulder surgery and more broadly in other kinds of tissue in the body.

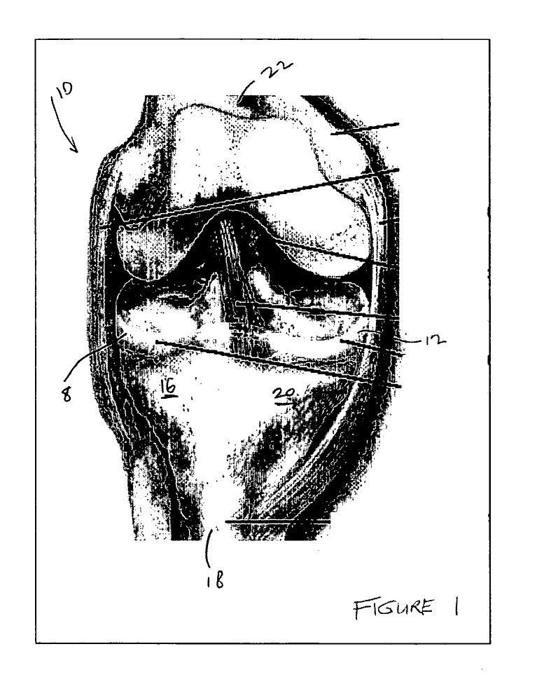

Focusing for the moment on the knee, as shown in FIG. 1, a meniscus 8 is a crescent-shaped piece of flexible cartilage. Menisci are found in both the human wrist and the human knee. There are two menisci in each knee 10, the medial meniscus 12 and lateral meniscus 8. The lateral meniscus rests atop the outer portion 16 of the tibia 18, more commonly referred to as the shinbone. The medial meniscus rests atop the inner portion 20 of the shinbone. Together, the two menisci act as a shock absorber between the tibia and femur 22, more commonly called the thighbone.

Meniscus tears result from either degenerative joint disease or awkwardly landing on and twisting the knee. Over time, wear and tear damage and unnatural motions degenerate the menisci, causing the degenerative joint disease. More specifically, a concentration of force from the femur isolated on one portion of the meniscus drives degeneration. Eventually this causes either a catastrophic failure or small tear of the medial meniscus or lateral meniscus.

Symptoms of meniscus tears include knee pain, swelling, locking, and sporadic failure of the knee. Meniscus tears are usually diagnosed by an orthopedist during a physical examination. Sometimes orthopedists and physicians use a magnetic resonance image (MRI) test to positively confirm the presence of meniscus tears. When doctors identify meniscus tears, they initially refrain from surgical procedures to allow the meniscus to heal on its own. If the tear does not heal on its own within about six weeks, it is unlikely that the meniscus injury will heal and arthroscopy is required. The arthroscopy allows doctors to locate the tear, identify its shape, and determine the appropriate course of action to repair or remove the meniscus.

Meniscus tears vary in shape, size, alignment, and location, as menisci fail differently under the various encountered stresses. Meniscus tear types include partial radial, complete radial, longitudinal, double longitudinal, flap, and horizontal cleavage tears. The most common type of tear that doctors repair is the bucket-handle tear.

The longitudinal meniscus tear 21 (shown in FIG. 2), typically involving the posterior section of the meniscus, is the second most common meniscus tear. The longitudinal meniscus tear occurs when a person runs or walks and abruptly changes direction with his knee bent, anchoring his foot to the ground, rotating the upper leg. The joint then traps the medial meniscus between the femur and tibia, pulling it towards the center and tearing it. The lateral meniscus is more mobile with less peripheral attachment and commonly tears when the knee suddenly extends, placing a sudden distraction force on the meniscus. The tear then propagates along the circumference of the meniscus.

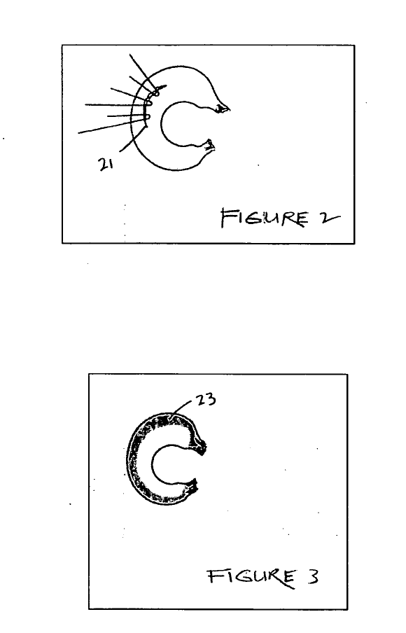

Whether a meniscus tear is repairable or whether menisectomy (meniscus removal) is necessary depends on the blood supply to the area surrounding the tear. As shown in FIG. 3, the outer one-third 23 of the meniscus is vascular, with sufficient blood supply to induce meniscus regeneration. However, the inner two-thirds of the meniscus are not vascular and will not regenerate. If a smooth, straight tear occurs on the outer third of the meniscus, then a series of several small sutures are usually sufficient to repair the tear. However, if the meniscus tear is jagged or located in the inner two-thirds of the meniscus, then menisectomy is required.

Removed portions of the meniscus do not grow back. Further joint degeneration often occurs if the damaged portion of the meniscus is not removed carefully. In the past, the entire meniscus was removed for all types of tears and patients had good knee function for many years. The precision of currently practiced arthroscopy allows doctors to reduce the area of the meniscus that needs to be removed. Reducing the amount of meniscus removed increases the amount of time the patient will have normal knee function after menisectomy.

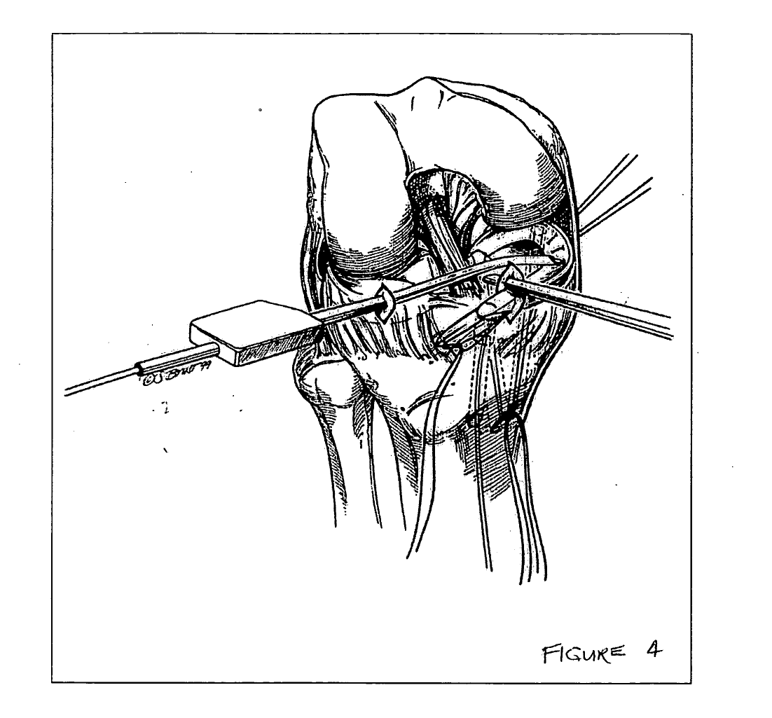

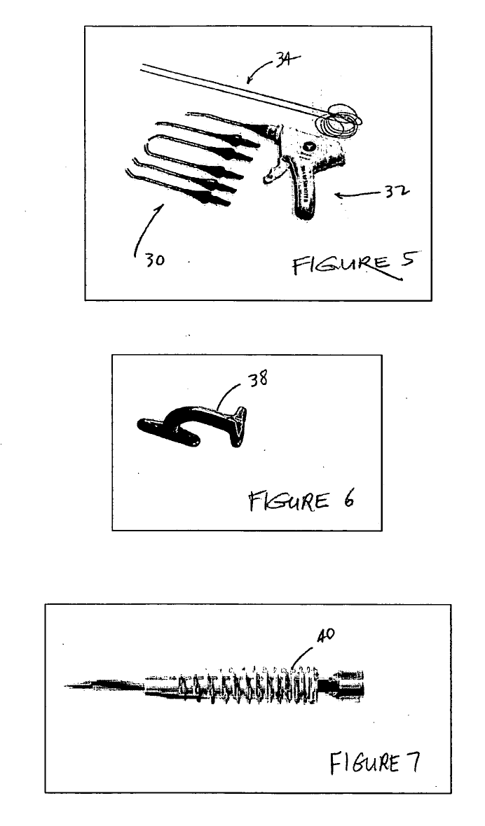

Manual meniscus repairs typically take up to two hours and require two highly skilled orthopedic surgeons to apply three to eight manual sutures to the meniscus. As shown in FIG. 4, in this procedure, a surgeon inserts an arthroscope through a quarter-inch incision made in the front of the knee. The knee is pumped full of surgical saline. The doctor uses the arthroscope to locate the tear on the meniscus. Next, two more incisions are made to facilitate the meniscus repair procedure. One small incision is made in the front of the knee, and another larger incision, around one-half inch, is made in the side of the knee.

A small metal tube called a cannula enters the knee through the second incision on the front of the knee to provide a path for suturing needles. Two flexible six-inch needles connected by suture material provide sutures for the torn meniscus. Each set of pre-threaded needles makes one suture and costs around $50 per set.

A metal retractor is inserted in the larger incision on the side of the knee to protect the vascular structures in the knee and help the second orthopedic surgeon find the needle after it passes through the meniscus.

Once the equipment is in place, the suturing process begins. Initially, the needle is manually forced through the cannula and then through the meniscus until it hits the retractor. Next, the second surgeon pulls the needle through the meniscus from the retractor incision. The needle is advanced a quarter inch at a time using forceps to avoid buckling the end of the flexible needle that extends out the top of the cannula. After the needle is carefully pulled all the way through the knee, the cannula is placed diagonally across the tear in the meniscus, and then the second needle is pushed through the meniscus and retrieved in the same fashion as the first. The suture is made diagonally across the tear to ensure that the suture material, typically PDS pr Ethibond (Johnson & Johnson, New Jersey), will not damage the longitudinal thin fibers of the meniscus. The second surgeon, working through the retractor incision, cuts off the two needles, ties a knot in the thread, and then slides the knot tight against the backside of the meniscus. This procedure is repeated to form additional knotted stitches along the length of the meniscus tear.

Various devices have also been developed for easier, faster stitching of body tissue and to improve the uniformity, strength, and effectiveness of the stitching.

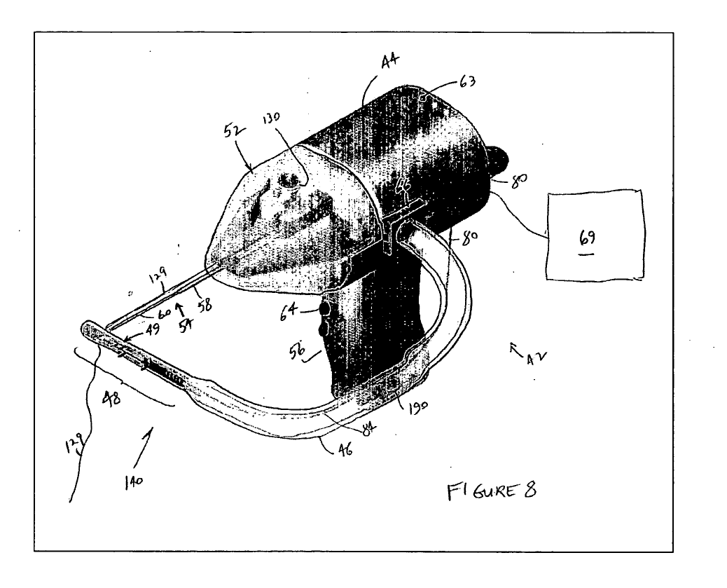

For example, Regenbio (located in Franklin Lakes, N.J.) developed a system to make manual suturing faster and easier. As shown in FIG. 5, cannulas 30 of various shapes are custom made to interface with a gun-type trigger mechanism 32. Trigger motion in the handheld portion of the system advances longer needles 34 through the cannulas and subsequently through the meniscus.

Another tool to repair torn menisci is produced by Mitek (located in Norwood, Mass.). The Mitek system eliminates the second incision required in the side of the knee. In this procedure, the doctor first measures the meniscus to determine the length of a needed fastener 38, shown in FIG. 6. Then, the fastener is attached to an applicator needle and incision device. The incision device has a hand trigger mechanism to deploy the fastener. The fastener is carefully pushed through the meniscus, and at the proper depth, the trigger is pulled, deploying the second end of the fastener.

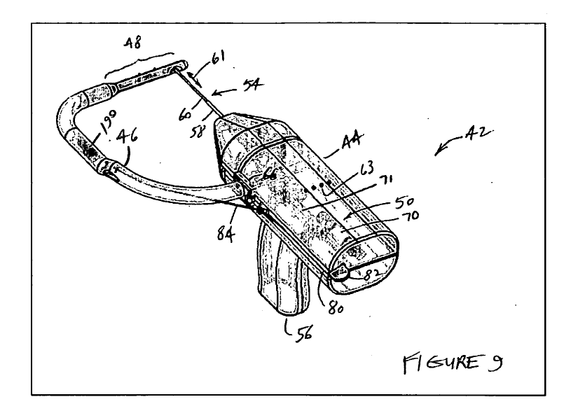

Mitek also makes a screw 40 shown in FIG. 7 to repair longitudinally torn menisci. The screw secures the two sides of the meniscus together. This procedure requires only two incisions in the front of the knee, eliminating the third incision in the side of the knee. Specially designed cannulas sheathe the screw while the doctor positions it. Once the cannula is properly positioned, the doctor forces the screw out with a specially designed screwdriver. Similar to conventional screws, the doctor turns the screwdriver clockwise to tighten the screw and counterclockwise to loosen or reposition the screw. The screws are designed to slowly bio-absorb after the meniscus is fully healed.

### SUMMARY

In general, in one aspect, the invention features an apparatus comprising a needle, a suture, stitching elements to cause the needle and the suture to form a stitch in a body tissue in response to a triggering event, and an element to receive the triggering event.

Implementations of the invention may include one or more of the following features. The suture includes a continuous suture used to form a succession of stitches in the body tissue in response to a succession of the triggering events. The stitch comprises a chain stitch. The needle includes a hole through which the suture is threaded. The stitching elements include a support to hold the needle on one side of the tissue and a receptor to receive the needle on the other side of the tissue after the needle has pierced the tissue, the receptor being held in a predetermined orientation and position relative to the support. The receptor includes a looper mechanism that has two loopers and a detector to detect if a stitch fails. The stitching elements are configured to perform a recovery procedure when the stitch fails. The support and the receptor are configured for use on a human knee. The stitching elements include a pneumatic system. A pneumatic cylinder is coupled to drive the needle. A pneumatic cylinder is coupled to drive the receptor. The element that receives the triggering event is configured to be activated by a human user or by a robotic user. The needle comprises a needle, a needle rod, and a cannula. A controller controls the stitching elements to operate in a sequence of steps to form the stitch. At least some of the stitching elements are removable from the apparatus.

In general, in another aspect, the invention features receiving a triggering event at a stitching device that has been placed in a position for forming a stitch in a body tissue, and forming a stitch in the body tissue in response to the triggering event.

Implementations of the invention may include one or more of the following features. After the completion of the stitch, another triggering event is awaited, and another stitch is formed in response to the other triggering event. The stitch is formed by piercing a previously formed loop of a suture and forming another loop. Automatically forming a stitch includes forcing a thread-bearing needle through the body tissue and forming a loop in the thread on a side of the body tissue opposite the entry point of the needle. The loop is formed by pulling at least one looper. The stitch is formed by a sequence of steps that include advancing a needle, partially retracting the needle, forming a loop in a thread borne by the needle, further retracting the needle, and advancing the needle through the loop. In implementations of the invention the body tissue may include knee tissue, for example, a meniscus.

In general, in another aspect, the invention features, positioning a stitching device in the vicinity of a body tissue to be stitched, performing a triggering event to cause the stitching device to form a complete stitch in response to the triggering event, repositioning the stitching device for forming another stitch, and performing another triggering event to cause the stitching device to form another complete stitch.

In general, in another aspect, the invention features piercing a body tissue using a needle bearing a suture, automatically piercing a loop previously formed in the suture, and automatically forming another loop in the suture.

In general, in another aspect, the invention features an apparatus comprising a suturing device having a handle and a needle for piercing a body tissue, a receptor positioned to receive a point of the needle after the needle has pierced the body tissue, and a spanning element configured to clear a knee in which the body tissue is located and to hold an axis of the needle and the receptor in fixed positions relative to one another.

In general, in another aspect, the invention features an apparatus comprising two masses of body tissue, and a chain-stitched suture penetrating both masses and tending to hold the two masses together. The two masses of body tissue comprise two parts of a meniscus.

In general, in another aspect, the invention features an apparatus comprising a suturing device comprising a needle, a handle, a suture, and a suture management system to manage the suture, in which at least portions of the needle, suture, and suture management system are removable from the handle and disposable.

In general, in another aspect, the invention features an apparatus comprising a suturing device to form sutures in tissue, the suturing device including a suture management system having controllable tensioning elements to selectively produce snug sutures or to reduce tension to permit a loop to be formed in the suture.

In general, in another aspect, the invention features, an apparatus comprising a receptor to form a loop in a suture for a chain stitch after a needle has carried the suture through a body tissue. The receptor includes a hooking mechanism, and a guide to direct motion of the hooking mechanism toward and away from the needle.

Implementations of the invention may include one or more of the following features. The hooking mechanism includes two hooks. The guide causes the two hooks to move closer together as they are moved toward the needle and to move farther apart as they are moved away from the needle. The guide includes a detector to detect a failure to form the loop in the suture. The detector includes a trip wire. The receptor is mounted on a housing, and the needle is supported on the housing in a predetermined orientation relative to the receptor. The receptor is mounted on a receptor arm that is removably attached to the housing by a releasable connector.

In general, in another aspect, the invention features apparatus that includes a pneumatic system to drive a needle and a receptor of a suturing device, the pneumatic system including separate pneumatic cylinders to respectively drive the needle and the receptor, and a controller to control the pneumatic system to perform a sequence of steps to form a chain stitch in a body tissue.

In general, in another aspect, the invention features apparatus that includes a thread management system for a suturing device, the thread management system including a frictional clutch arranged to apply tension to thread as it is fed to a needle of the suturing device, and a brake arranged to relieve tension on the thread during a portion of a stitching operation on a body tissue.

Implementations of the invention may include one or more of the following features. The frictional clutch includes a torsional spring. The brake includes a cam and a backstop. The brake is automatically triggered by motion of the needle. The thread is held on a bobbin and the frictional clutch operates on the bobbin.

Among the advantages of what is described here are one or more of the following. Stitches can be placed quickly, safely, and easily. The stitches are uniform, secure, and neat. A single user can create stitches without assistance. The device may be autoclaved. Some parts are disposable. The chances for needle sticks into the hands of the user are reduced compared to other methods. The resulting stitches are as strong and precise as manual hand sutures.

Other advantages and features will become apparent from the following description and from the claims.

### DESCRIPTION

We first briefly describe the figures.

FIG. 1 shows an anatomical view of a right knee.

FIG. 2 shows a longitudinal meniscus tear.

FIG. 3 shows a repairable portion of a meniscus.

FIG. 4 shows a meniscus repair instrument and incision locations on a knee.

FIG. 5 shows a meniscus repair tool.

FIG. 6 shows a meniscus repair fastener.

FIG. 7 shows a meniscus repair screw.

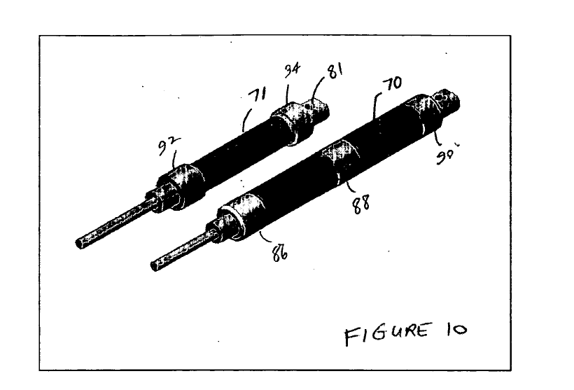

FIG. 8 shows a three-dimensional view of a suturing device.

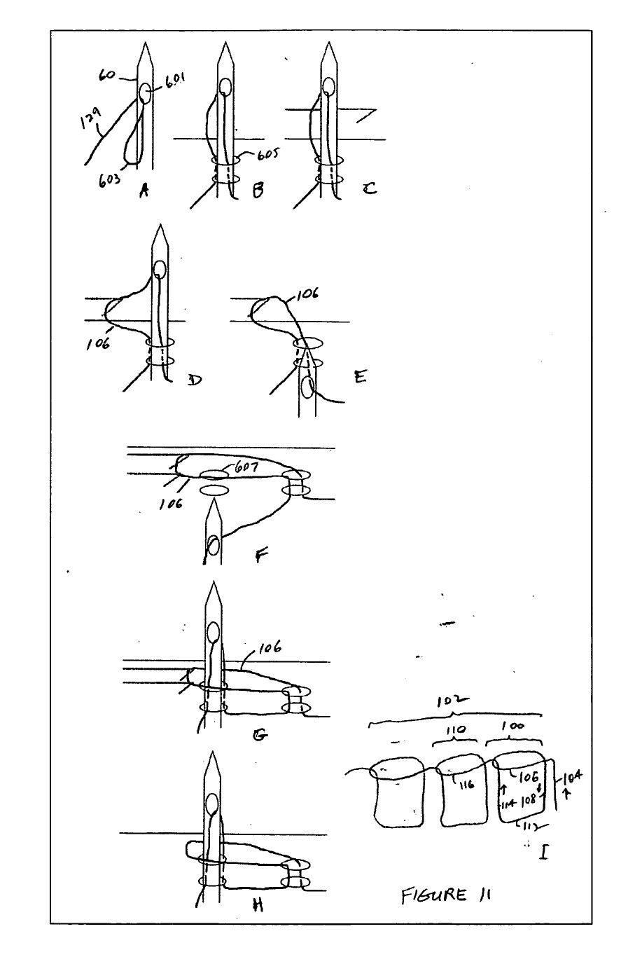

FIG. 9 shows a three-dimensional view of a suturing device.

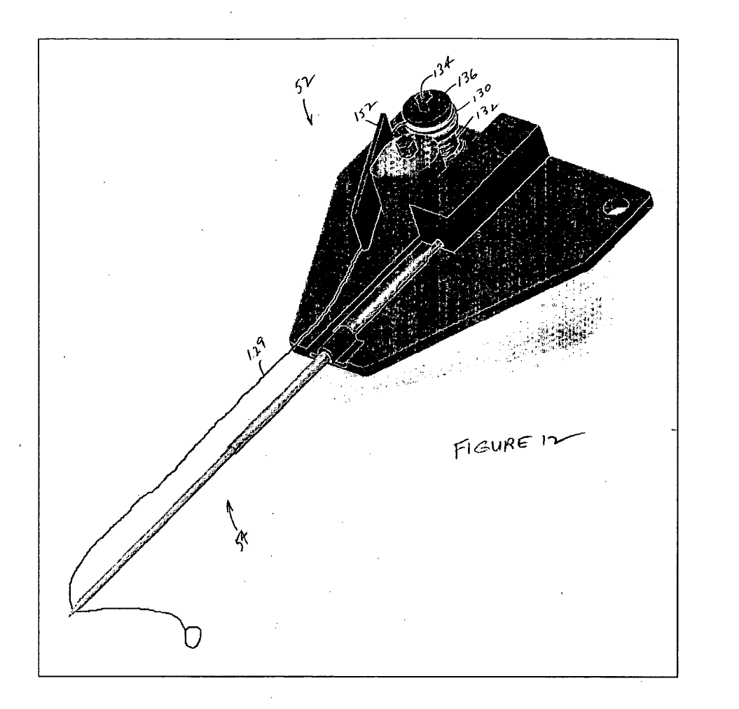

FIG. 10 shows pneumatic cylinders.

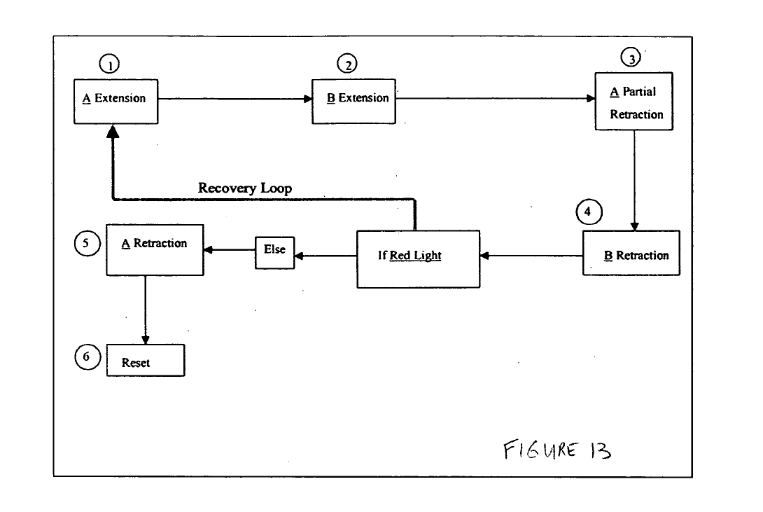

FIG. 11 shows chain stitches.

FIG. 12 shows a thread management system.

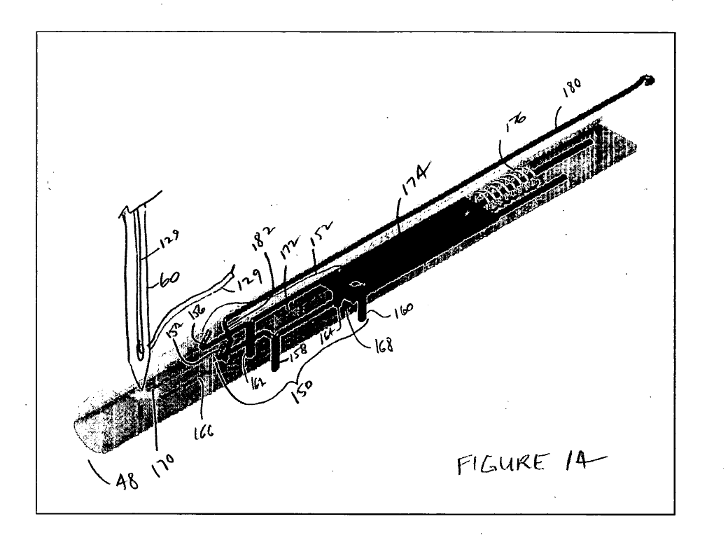

FIG. 13 shows a pneumatic sequence.

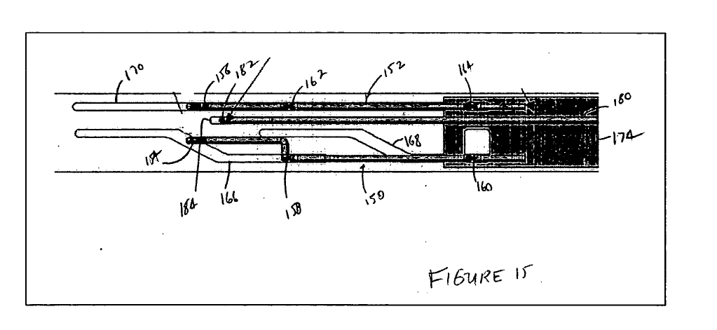

FIGS. 14 and 15 show receptors.

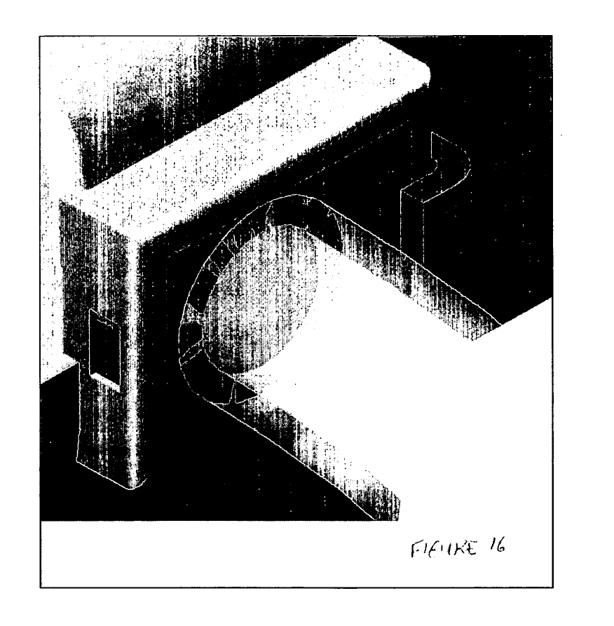

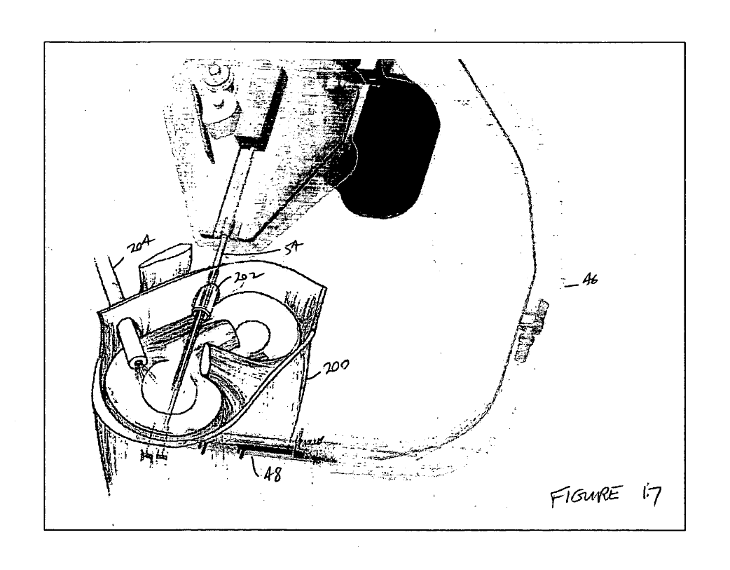

FIG. 16 shows a receptor arm connector.

FIG. 17 shows a suturing device in use.

Without implying any limitation on the scope of the techniques and devices described below or their uses, we focus on repair of a meniscus as an example.

Housing and Systems

As shown in FIGS. 8 and 9 (the two figures represent slightly different versions; in FIG. 9, the housing and the receptor arm are shown as being transparent; in FIG. 8, the receptor arm is shown as being transparent), an automatic suturing device 42 includes a main housing 44 and a receptor arm 46 that attaches to the housing. The receptor arm has a contour that accommodates the geometry of a human knee. The end of the receptor arm 46 holds a receptor 48 that includes two loopers 49 that grab the thread from the needle to form a loop that becomes part of each conventional chain stitch in a series of chain stitches. The main housing incorporates a pneumatic system 50, a thread management system 52, and a needle system 54. The main housing is supported by an ergonomic handle 56. The needle system 54 includes a cannula 58 through which a needle 60 oscillates 61 during the formation of the chain stitches.

The components within the main housing are accommodated within the flask-like shape of its outer casing. The handle has finger buttons 60 for control of the suturing functions, and the housing includes signal lights 63 to indicate the state of the suturing tool and suturing functions. The receptor arm can be attached to and detached from the main housing using a ski-binding type clip 66. The needle system, thread management system, and needle system are all attached to the main housing when the suturing begins.

Pneumatic System

The pneumatic system includes two cylinders 70, 71 (FIG. 10) that respectively power the needle 60 and the receptor 48. The cylinders are controlled by valves, switches, and pneumatic logic to achieve an operating sequence that creates a series of chain stitches. The pneumatic logic (not contained in the main housing and not shown) is attached by hoses to the main housing. Separating the pneumatic logic from the main housing reduces the weight of the handheld portion of the device. The components of the pneumatic system can be provided by a variety of manufacturers, for example, Sprague Air Controls, Inc., located in Hingham, Mass., based on the designed operating sequence and on defined pneumatic specifications derived from testing on human cadaver knees.

The minimum force that the needle cylinder 70 must exert on the needle to penetrate the meniscus is expected to be typically no more than about 4 g. Tension on the thread carried by the needle—estimated at 1.5 kgf (14.71 N)—also affects the operation of the pneumatic cylinders. The needle cylinder force required to penetrate the meniscus is the sum of the penetration force and the thread tension and translates into a pressure of 46,948 Pa (2.168 psi).

The needle cylinder has three positions to perform the following sequence (the dimensions are examples only): extend fully (44.5 mm); retract 5.5 to 6.5 mm to allow the receptor to catch the suture loop; and retract the needle fully to its original position. The needle cylinder operates directly on the needle through the front wall of the main housing.

The receptor cylinder need be no larger than the needle cylinder because it requires a similar stroke length and simpler operating specifications. The receptor cylinder operates the receptor through a stiff wire 80 that passes through the rear wall of the main housing, wraps around a pulley 82 and into a metal-reinforced plastic flexi-cable 84 (similar to those used in lawnmower throttles). The flexi-cable passes into and along an internal channel of the receptor arm to the receptor. One end of the wire 80 within the main housing attaches to the piston at a hole 81 (FIG. 10) drilled through a shaft of the piston perpendicular to the axis of the piston. A crimped soft metal coupling attaches the receptor to the other end of the wire 80.

Referring again to FIG. 10, because the needle cylinder has three ports 86, 88, 90, it needs three control valves to control the positioning of the stroke. The receptor cylinder has two ports 92, 94 and needs two control valves, because its strokes are simpler. This five-valve system may be electronically controlled by a computer chip (not shown) programmed to execute the exact sequence of both cylinders' motion.

Chain Stitching

As shown in FIG. 11, step I, each chain stitch 100 in a series of chain stitches 102 begins with a stroke of the needle that delivers the thread along an upward trajectory 104 in the figure. Next, the needle is retracted partway and a loop of thread 106 is pulled from the tip of the needle and held by the receptor. Then the needle is withdrawn fully, along a downward trajectory 108 in the figure, while the receptor retains the loop of thread on the backside of the meniscus and the thread runs through the hole 601 at the end of the needle. As part of the next stitch 110, the needle is moved to a new position 112 and then extended along a new upward trajectory 114 through the previously pulled loop. The previous loop of thread is released by the retractor and a new loop 116 is pulled from the thread by the retractor, essentially finishing the first stitch 100, and the process repeats. Additional detail is shown in steps A through H of FIG. 11. In step A, prior to the first stitch, the needle holds the thread through hole 601 and a knotted loop 603 on the free end keeps the thread from pulling through the hole. The needle is driven upward to form a hole 605 in the meniscus. In steps B, C, and D the retractors begin to pull a loop 106 and in step E the needle is withdrawn partially. The two retractors pull the loop and the needle is repositioned to a new location for piercing a second hole 607 in the meniscus (step F). In step G the needle has pushed the thread up through the loop and in step H the retractors have released the loop and a new stitching sequence is ready to begin.

Thread Management

As shown in FIG. 12, the thread management system 52 controls feeding and tension in the thread 129 during suturing. During most of the operating sequence, the thread management system must provide sufficient tension to ensure the sutures are snug and that the thread is not tangled. The initial assumed tension to maintain in the thread is, for example, 14.71 N. When the needle is fully extended, the tension in the thread must be released to allow a suitable loop to form as the needle retracts 5.5 to 6.5 mm. To accomplish this, the thread management system may operate much as a tape measure, using a torsional spring to maintain tension and a brake to relieve the tension. The thread management system also has a role in a recovery sequence that occurs after the device determines whether the stitch was successful or if another attempt is necessary.

The thread (medical grade and FDA approved) is stored on a bobbin 132 similar to those used in a traditional sewing machine. The bobbin rotates on a bobbin post 134 and is held in contact with a torsion disk 136 by a torsional compression spring 130 that also rotates around the bobbin post. The compression spring applies pressure between the bobbin and torsion disk creating a friction clutch.

The friction clutch applies constant tension on the bobbin while it unwinds, but allows the bobbin to slip as thread is required during suturing. The thread is fed to the needle during the forward stroke of the needle cylinder. Adjustment of the clutch delivers the desired consistent tension during thread-feeding. If the surgeon needs to apply or release tension during the suturing, a manual thumb dial (not shown) provides access for manual tension adjustments.

While the receptor loops the thread, the tension in the thread is relieved by a mechanical brake in the form of a cam 149 and wedge. The cam is engaged by a wedge (not shown) on the needle rod during the maximum forward stroke. The cam and wedge operate by direct contact; as the wedge is driven forward; it engages the cam and causes it to rotate on an offset axis. The cam pinches the thread on a solid backstop 152 attached to a compression spring, thus holding the thread and preventing tension from being applied. When the needle rod retracts, the wedge disengages the cam from the backstop, releasing the thread, and tension is returned to the stitch.

Using a simple friction clutch, torsion spring, and wedge/cam brake for the thread management system reduces the chance for failure and makes initial test adjustments easy.

The Needle System

The needle system includes a cannula, a needle rod that rides within the cannula and a needle on the end of the needle rod. The needle must withstand a compressive load during the forward stroke, and the needle tip, which is pierced by a hole for the thread, is subjected to the largest stress concentrations. It is important to prevent the minute needle tip from breaking off into the body, where it will be nearly impossible to retrieve. Changing the shape of the needle, hole, and thread may achieve a stronger needle. Using a polymer material similar to rubber having a larger fracture toughness may be desirable. Testing may be needed to determine the best diameter for the needle and for the thread hole.

The needle cannula, made of stainless steel or titanium tube, may be the same kind used in manual suturing, protects the patient from the movement of the needle rod, and provides structural support for the needle rod so it remains aligned on its intended axis. When attaching the cannula to the housing, the orthopedist must first slide the needle into the cannula, as the needle must be attached to the piston of its needle cylinder prior to the cannula being attached to the housing. A cannula connector within the main casing (made, for example, of surgical stainless steel) integrates the standard straight cannula with the suturing device. The cannula is held by tightening a small knob. The needle/needle rod assembly with pre-attached thread must be replaced after each procedure for sanitation purposes.

The needle rod has a thicker diameter than the needle to improve the overall strength of the needle system. The diameter of the rod may be, for example, 1.85 mm, only 0.15 mm smaller than the inner diameter of an example cannula. The thread may be fed along a v-shaped trough machined into the surface of the needle rod. The trough ensures the thread does not get twisted within the cannula. The only lubrication between the needle rod and inner surface of the cannula may be sterile saline, as other fluids may damage the body.

One end of the needle rod is attached to the needle. The other end of the needle rod attaches to the piston. In one example, the end of the piston arm is threaded and a small adapter screws on to allow the needle rod assembly to slide in and tighten using threads. Another possibility for connection would be to weld a larger adapter to the piston end of the needle rod and then secure the larger portion.

Suturing Sequence

The suturing sequence is designed to achieve the desired chain stitch. For safety, a whisker valve is incorporated in the pneumatic system. If the suturing device fails to complete the partial retraction step of the needle cylinder, the suturing device will return to the beginning of the sequence, thus avoiding stitches coming apart due to an incomplete stitching cycle. As shown in FIG. 13, a complete sequence for the two pneumatic cylinders is as follows (A refers to the needle cylinder and B refers to the receptor cylinder):
1. Extend A (in response to the pressing of a handle button by the orthopedist).
2. Extend B.
3. Partially retract A (to intermediate position to permit the creation of the loop in the thread).
4. Retract B (loopers catch the loop of thread). If loop of thread is not caught by the loopers, the whisker valve will not trigger the rest of the sequence. Return to the beginning and start cycle again. Otherwise, continue on to Step 5
5. Completely retract A (to reset position).
6. Reset in preparation for next stitch.

There is no need for special functions for the initial suture or the final suture. For the final suture, the user presses an off button, takes apart the device, and ties off the final stitch.

The suturing process is controlled by a controller 69 (FIG. 8; in the form of software, hardware, or a combination of software and hardware). Valves, relays, buttons, and toggle switches provide input to the controller to produce a chain stitch, a commonly used sewing stitch. The controller is arranged to perform, for example, the following sequence to complete each stitch. The surgeon determines when to begin each stitch by pressing the “suture” button:
1. Detect pressing of suture button.
2. Illuminate red busy light.
3. Extend needle cylinder to extend needle/needle rod to its terminal point.
4. Extend receptor cylinder to extend receptor to its terminal point.
5. Release tension on thread by partially retracting needle cylinder.
6. Retract receptor cylinder to cause loopers to return to start position, grabbing loop, and tripping a toggle switch.
7. Re-engage thread tension.
8. Retract needle cylinder to withdraw needle to start position.
9. Extinguish red busy light.
10. Illuminate green ready light.

If a stitch fails, a recovery loop algorithm may be implemented as follows:
1. If toggle switch in receptor signals a successful stitch, continue suturing sequence, otherwise flash red light.
2. Re-engage tension.
3. Extend needle to terminal point.
4. Extend receptor cylinder to push loopers to terminal point.
5. Disengage thread tension.
6. Retract needle partially.
7. Withdraw receptor cylinder to return loopers to original position, grabbing thread and switching toggle switch.
8. If toggle switch signals a successful stitch, change flashing red light to solid red light, continue to step 7 of suturing process, otherwise return to step one of recovery loop.

The receptor arm is the round curved structure containing the needle receptor at its end. The structure wraps around the knee so the needle receptor always remains directly opposite the needle. To preserve precise alignment necessary for the needle and receptor, the arm must not bend.

Receptor Arm and Receptor

Referring again to FIG. 8, the receptor arm is generally semi-circular to accommodate a variety of knee geometries, including knees that are deformed because of injuries, arthritis, illness, or accident.

The receptor arm is a cylindrical thin-walled tube. In one example, the outside diameter of the tube is 12.7 mm, and the inner diameter of the tube is 11.7 mm. The receptor arm could be made of surgical stainless steel or titanium, for example.

The external radius of the receptor arm, in one example, is 141.3 mm, which accommodates larger than average knees. The receptor arm radius accounts for increased knee diameter during the procedure as a result of swelling from the sterile saline solution.

The receptor is supported at the free end 140 of the receptor arm. During each stitch, the pair of loopers in the receptor catch the suture from the tip of the needle and create a loop. There are two loopers to assure that a large enough loop is created. The hook ends of the loopers are oriented in the same direction as the needle at its furthest extension to reduce the chance of the looper endsgrabbing the needle rather than the thread, which could jam the device or break the needle. At the end of each stitch, the loopers are retracted to release the prior loop and to form a new loop for the next stitch.

In one example, the receptor 48 is designed to fit into a 0.4-inch inside diameter cylinder at the end of the receptor arm. As shown in FIGS. 14 and 15, each of the two loopers 150, 152 has an angled hook 154, 156 at one end, a pair of short legs 158, 160, and 162, 164. The wall of the receptor arm is cut to define tracks 166, 168 and 170, 172 that guide the legs as they move back and forth, keeping the loopers parallel and moving in the intended directions.

A flat slide 174 is used to drive the loopers synchronously back and forth. For this purpose, the rear foot of each looper passes through a hole in the slide. The loopers may be made of bent music wire 0.075 inch thick. The two tracks 166, 168 have offset contours that cause the hook of looper 150 to move sideways toward and into contact with looper 152 when the two loopers are pushed by the slide toward the needle tip and to separate the hooks of the loopers when they are withdrawn away from the needle, thus expanding the size of the loop.

The transfer of force between the receptor cylinder in the main housing and the loopers in the receptor is achieved, in one example, by a pull-pull system. In the pull-pull system, wire 80 (FIG. 9) is pulled by the cylinder and in turn pulls the slide to cause the loopers to form the loop. A spring 176 then pushes the slide to return the loopers for the next sequence. The pull-pull system is more flexible, lighter, and more efficient than a pull-push system would be.

In one example, the needle receptor is a box-shaped structure 70 mm long×12.7 mm wide×12.7 mm deep with a 1 mm wall thickness. On the inner wall of the receptor end is a hole allowing the needle to enter.

As shown in FIG. 16, the receptor arm 46 easily attaches and detaches from the main housing using a plastic ski-binding type clip 66. The ease of attaching and detaching the receptor arm to the main housing is important as the receptor is attached after the main housing has already been secured to the cannula and needle.

Recovery Sequence; Trip Mechanism

During the formation of a loop, if only one looper grabs the thread, the loop will be off center and therefore not positioned to receive the needle when the needle returns to the receptor to finish the stitch. If neither looper has grabbed the thread, the stitch will be lost and the user will have to start the stitch again. In either event, to detect and handle these failed stitches, a stiff trip wire 180 (FIGS. 14, 15) includes a leg 182 that rides in a track 184 between the looper hooks. The trip wire is not driven by the slide 174 but rather remains with its small leg in a fixed position along the length of its track until the thread loop is pulled back by the looper hooks and forces the trip wire back. This trip wire is linked to a whisker valve 201 (FIG. 17) in the receptor arm so that when the thread loop pushes the wire the valve will be tripped and will signal the controller that a stitch has been successful, triggering the rest of the stitching sequence. Otherwise, the controller initiates the recovery sequence.

FIG. 17 shows the suturing device in use. The needle system 54 has been inserted into the knee 200 through an incision and is surrounded by a temporary sleeve 202. An arthroscope 204 has been inserted through a second incision for illumination and visibility. The receptor arm remains outside the knee joint deep to the skin and superficial fascia. Each stitch penetrates through the meniscus and through the capsule. After each stitch, the user moves the device to a new position for the next stitch. At the end of the series of stitches, the final stitch is tied off on the outside of the knee and the free end of the thread is cut and pulled out along with the needle system.

Other implementations are within the scope of the following claims.

For example, although the chain stitch is described above, other kinds of stitches may also be suitable, and the elements needed to make the other kinds of stitches may be different from the ones described above.

Other shapes of receptor arms and needle systems may be provided for use with other cannula shapes. Different configurations would allow the respective cannulas to reach different portions of the meniscus. By substituting other geometry cannulas and other geometry receptor arms to match the cannulas, the suturing device can be specialized for a variety of uses. A single machine having interchangeable arms, cannulas, and needle rods may be used to reach any desired position on the meniscus or other part of the body.

Although stainless steel and titanium have been mentioned as possible materials for parts of the suture device, polymers, composites, and ceramics might also be used for advanced control, valve, and sensor parts.

A second arm can be provided on the other side for the housing to accommodate the lateral meniscus.

The ergonomics and shape of the housing may be adjusted to accommodate the preferences of different users.

In other applications, the suturing device could be held by and controlled by a robotic machine that would be under the indirect control of a professional. Such a robotic machine could position the suturing device relative to the patient, trigger a stitch, reposition the suturing device, trigger another stitch, and so on in a sequence of actions that occur automatically.

The needle need not be driven by pneumatic drivers; other approaches, including hydraulic, electronic, and manual, could be used.

Additional information, details, and alternatives are set forth in Appendices A through H and incorporated by reference.

1. Apparatus comprising
a needle,
a suture,
stitching elements to cause the needle and the suture to automatically form a stitch in a body tissue in response to a triggering event, and
an element to receive the triggering event.

2. The apparatus of claim 1 in which the suture comprises a continuous suture to form a succession of stitches in the body tissue in response to a succession of the triggering events.

3. The apparatus of claim 1 in which the stitch comprises a chain stitch.

4. The apparatus of claim 1 in which the needle includes a hole through which the suture is threaded.

5. The apparatus of claim 1 in which the stitching elements include a support to hold the needle on one side of the tissue and a receptor to receive the needle on the other side of the tissue after the needle has pierced the tissue, the receptor being held in a predetermined orientation and position relative to the support.

6. The apparatus of claim 5 in which the receptor includes a looper mechanism.

7. The apparatus of claim 6 in which the looper mechanism includes two loopers.

8. The apparatus of claim 5 in which the receptor includes a detector to detect if a stitch fails.

9. The apparatus of claim 8 in which the stitching elements are configured to perform a recovery procedure when the stitch fails.

10. The apparatus of claim 5 in which the support and the receptor are configured for use on a human knee.

11. The apparatus of claim 1 in which the stitching elements include a pneumatic system.

12. The apparatus of claim 11 in which the pneumatic system includes a pneumatic cylinder coupled to drive the needle.

13. The apparatus of claim 5 in the stitching elements include a pneumatic cylinder coupled to drive the receptor.

14. The apparatus of claim 1 in which the element that receives the triggering event is configured to be activated by a human user.

15. The apparatus of claim 1 in which the element that receives the triggering event is configured to be activated by a robotic user.

16. The apparatus of claim 1 in which the needle comprises a needle, a needle rod, and a cannula.

17. The apparatus of claim 1 including a controller to control the stitching elements to operate in a sequence of steps to form the stitch.

18. The apparatus of claim 1 in which at least some of the stitching elements are removable from the apparatus.

19. A method comprising
receiving a triggering event with respect to a stitching device that has been placed in a position for forming a stitch in a body tissue, and
automatically forming a stitch in the body tissue in response to the triggering event.

20. The method of claim 19 also including
after the completion of the stitch, awaiting another triggering event, and
forming another stitch in response to the other triggering event.

21. The method of claim 19 comprising
forming the stitch by piercing a previously formed loop of a suture and forming another loop.

22. The method of claim 19 in which automatically forming a stitch includes forcing a thread-bearing needle through the body tissue.

23. The method of claim 19 in which automatically forming a stitch includes forming a loop in the thread on a side of the body tissue opposite the entry point of the needle.

24. The method of claim 23 in which the loop is formed by pulling at least one looper.

25. The method of claim 19 in which the stitch is formed by a sequence of steps that include advancing a needle, partially retracting the needle, forming a loop in a thread borne by the needle, further retracting the needle, and advancing the needle through the loop.

26. The method of claim 19 in which the body tissue comprises knee tissue.

27. The method of claim 26 in which the knee tissue comprises a meniscus.

28. A method comprising
positioning a stitching device in the vicinity of a body tissue to be stitched,
performing a triggering event to cause the stitching device to automatically form a stitch in response to the triggering event,
repositioning the stitching device for forming another stitch, and
performing another triggering event to cause the stitching device to automatically form another stitch.

29. A method comprising
piercing a body tissue using a needle bearing a suture,
automatically piercing a loop previously formed in the suture, and
automatically forming another loop in the suture.

30. Apparatus comprising
a suturing device comprising
a handle for holding the device and a needle for piercing a body tissue,
a receptor positioned to receive a point of the needle after the needle has pierced the body tissue, and
a spanning element configured to clear a knee in which the body tissue is located and to hold an axis of the needle and the receptor in fixed positions relative to one another.

31. Apparatus comprising
two masses of body tissue, and
a chain-stitched suture penetrating both masses and tending to hold the two masses together.

32. The apparatus of claim 31 in which the two masses of body tissue comprise two parts of a meniscus.

33. Apparatus comprising
a suturing device comprising
a needle,
a handle,
a suture,
a suture management system to manage the suture,
at least portions of the needle, suture, and the suture management system being removable from the handle and disposable.

34. Apparatus comprising
a suturing device to form sutures in tissue, the suturing device including
a suture management system having controllable tensioning elements to selectively produce snug sutures or to reduce tension to permit a loop to be formed in the suture.

35. Apparatus comprising
a receptor to form a loop in a suture for a chain stitch after a needle has carried the suture through a body tissue, the receptor including
a hooking mechanism, and
a guide to direct motion of the hooking mechanism toward and away from the needle.

36. The apparatus of claim 35 in which the hooking mechanism comprises two hooks.

37. The apparatus of claim 36 in which the guide causes the two hooks to move closer together as they are moved toward the needle and to move farther apart as they are moved away from the needle.

38. The apparatus of claim 35 in which the guide includes a detector to detect a failure to form the loop in the suture.

39. The apparatus of claim 35 in which the detector comprises a trip wire.

40. The apparatus of claim 35 in which the receptor is mounted on a housing, and the needle is supported on the housing in a predetermined orientation relative to the receptor.

41. The apparatus of claim 40 in which the receptor is mounted on a receptor arm that is removably attached to the housing by a releasable connector.

42. Apparatus comprising
a pneumatic system to drive a needle and a receptor of a suturing device, the pneumatic system including separate pneumatic cylinders to respectively drive the needle and the receptor, and
a controller to control the pneumatic system to perform a sequence of steps to form a chain stitch in a body tissue.

43. Apparatus comprising
a thread management system for a suturing device, the thread management system including a frictional clutch arranged to apply tension to thread as it is fed to a needle of the suturing device, and a brake arranged to relieve tension on the thread during a portion of a stitching operation on a body tissue.

44. The apparatus of claim 43 in which the frictional clutch includes a torsional spring.

45. The apparatus of claim 43 in which the brake includes a cam and a backstop.

46. The apparatus of claim 43 in which the brake is automatically triggered by motion of the needle.

47. The apparatus of claim 43 in which the thread is held on a bobbin and the frictional clutch operates on the bobbin.
